(19) United States
(12) Patent Application Publication
(10) Pub. No.: US 2019/0292524 A1
(43) Pub. Date: Sep. 26, 2019

(54) METHODS FOR CANCER STEM CELL (CSC) EXPANSION

(71) Applicant: TRANSGENEX NANOBIOTECH, INC., Tampa, FL (US)

(72) Inventors: Rajesh NAIR, Tampa, FL (US); Mazen HANNA, Tampa, FL (US); Subhra MOHAPATRA, Lutz, FL (US); Shyam MOHAPATRA, Lutz, FL (US); Ryan GREEN, New Port Richey, FL (US)

(21) Appl. No.: 16/339,837

(22) PCT Filed: Oct. 6, 2017
(86) PCT No.: PCT/US2017/055530

§ 371 Date: Apr. 5, 2019

Related U.S. Application Data
(60) Provisional application No. 62/405,187, filed on Oct. 6, 2016.

(51) Int. Cl. C12N 5/095; C12N 5/00; C12N 15/113; C12Q 1/02

(52) U.S. Cl. CPC C12N 5/0695; C12N 5/0068; C12N 5/0062; C12N 15/1135; C12Q 1/025; C12N 2533/00; C12N 2513/00; C12N 2533/90; C12N 2310/14; C12N 2501/15; C12N 2533/40

(57) ABSTRACT

The invention relates to the methods to increase populations of cancer stem cells (CSCs), including human CSCs, using, for example, a FiSS™ (fiber-inspired smart scaffold) platform, a scaffold for cell culture comprising an electrospun mixture of poly(lactic-co-glycolic acid) (PLGA) and a block copolymer of polylactic acid (PLA) and monomethoxypolyethylene glycol (mPEG). As an example, we demonstrated that MCF-7 cells grown on FiSScsc developed into well-formed single-cell tumoroids (SCTs), showing a ˜3-fold increase in the cancer stem cell (CSC) population versus similar-passage cells grown as monolayers. This increase was further potentiated when the first-generation tumoroids were used to grow second- and third-generation tumoroids. Additionally, we scaled-up the cell culturing protocol from, for example, a 96-well plate to, for example, a 6-well plate, with no loss in the induction of CSCs. We also sorted and froze CSC-enriched cells and successfully thawed them again to grow tumoroids, while maintaining the CSC population.

This application claims priority to U.S. Application No. 62/405,187, filed Oct. 6, 2016, which is incorporated by reference.

### SUMMARY OF THE INVENTION

The present invention describes methods to increase the population of cancer stem cells (CSCs) using, for example, a FiSS™ (fiber-inspired smart scaffold) platform, a scaffold for cell culture comprising an electrospun mixture of poly(lactic-co-glycolic acid) (PLGA) and a block copolymer of polylactic acid (PLA) and monomethoxypolyethylene glycol (mPEG). In one embodiment, regular growth medium is used to grow first-generation tumoroids, and second-generation tumoroids from the first-generation tumoroids. At the end of each generation, the resulting tumoroids are processed and the cells analyzed for stem cell markers (e.g., CD44^{high}/CD44⁺ and CD24^{low}/CD24⁻), e.g., by flow cytometry. Surprisingly, the tumoroids have an ^{˜}3-fold increase in CSCs compared to the cancer cells used to grow the tumoroids.

In another embodiment, regular growth medium is used supplemented with cobalt chloride (CoCl₂) to mimic hypoxia in the tumoroids. Alternatively, cobalt chloride is infused into the scaffold matrix to ensure sustained hypoxic conditions for first-generation tumoroids growing on the scaffold.

The increase in CSCs is further, and unexpectedly, potentiated in the second-generation tumoroids, where an ^{˜}10-fold increase in CSCs was observed. In another embodiment, tumoroids are grown on cobalt chloride-infused scaffolds, resulting in larger first-generation tumoroids that show a trend towards increased CSCs compared with tumoroids grown on regular scaffolds.

In a further embodiment, the CSC population is further increased by culturing the tumoroids in conditioned medium (CM) collected from primary cancer-associated fibroblasts (CAFs) and myeloid-derived suppressor cells (MDSCs) from human peripheral blood.

In another embodiment, tumoroid culture conditions are expanded from, smaller well format, for example, a 96-well format, to a larger well format, for example, a 6-well format tissue culture dish, to increase the yield of CSCs (by ^{˜}30-fold), while maintaining the ability for CSC expansion.

In yet another embodiment, the CSCs are expanded to stored.

### BACKGROUND OF THE INVENTION

Cancers continue to constitute a major cause of morbidity and mortality worldwide. Traditional therapies often cannot completely eradicate tumors, prevent cancer recurrence, or prevent metastasis in lung cancer patients. Recently, in some cases, these failures in effectively treating cancers have been attributed to cancer stem cells (CSCs), which have properties of self-renewal, tumor initiation, and tumor maintenance, and are considered a major cause of mortality after relapse following treatment.

While chemotherapy and other conventional cancer therapies may be more effective at killing bulk tumor cells, CSCs may manage to escape and seed new tumor growth, due to the survival of quiescent CSCs (Clarke et al. (2006) Cancer Res. 66, 9339-44; Reya et al. (2001) Nature 414, 105-11). With growing evidence supporting the role of CSCs in tumorigenesis (Gupta et al. (2009) Nat. Med. 15, 1010-12), tumor heterogeneity (Meacham & Morrison (2013) Nature 501, 328-37), resistance to chemotherapeutic and radiation therapies (Li et al. (2008) J. Natl. Cancer Inst. 100, 672-9; Diehn et al. (2009) Nature 458, 780-3), and the metastatic phenotype (Shiozawa et al. (2013) Pharmacol. Ther. 138, 285-93), the development of specific therapies that target CSCs holds promise for improving the survival and quality of life for cancer patients, especially those with metastatic disease (Takebe et al. (2011) Nat. Rev. Clin. Oncol. 8, 97-106; Dalerba & Clarke (2007) Cell Stem Cell 1, 241-2).

Thus, there is a continuing and urgent need for the development of novel therapeutic agents that target CSCs, specifically agents that target CSC self-renewal, regeneration, and differentiation processes. These agents, such as small molecules or biologics, should be designed to target CSCs, CSC-related biomarkers, and CSC pathways that affect fundamental processes associated with carcinogenesis, tumor progression, maintenance, recurrence, and metastasis.

The maintenance of CSCs is regulated by their microenvironment. Thus, cell-extracellular matrix (ECM) interactions and cell-cell interactions can play important roles in stem cell reprogramming. The progression of CSCs to tumors depends on the tumor microenvironment or stroma that includes the ECM (e.g., collagen, fibronectin, laminin), endothelial cells, cancer-associated fibroblasts (CAFs), and immune cells (e.g., macrophages, neutrophils, lymphocytes). The tumor microenvironment induces activation of the epithelial-to-mesenchymal transition (EMT) in numerous cancer cells, including lung, colorectal, pancreatic, prostate, ovarian, and breast cancers (Polyak & Weinberg (2009) Nat. Rev. Cancer 9, 265-73).

Sustained activation of signal transduction showed that pathways involving hedgehog, epidermal growth factor receptor (EGFR), Wnt/β-catenin, Notch, transforming growth factor-β (TGF-β)/TGF-β receptors, and/or stromal cell-derived factor-1 (SDF-1)/CXC chemokine receptor 4 (CXCR4) play important roles in the high self-renewal potential, survival, invasion, and metastasis of CSCs (Takebe et al. (2011) Nat. Rev. Clin. Oncol. 8, 97-106; Singh et al. (2012) Mol. Cancer 11, 73). Transcription factors, such as Sox2, c-Myc, Oct4, Nanog, Klf-4, and Lin-28, also play important roles in the self-renewal of embryonic stem cells (Kim et al. (2008) Cell 132, 1049-61) and CSCs (Chiou et al. (2010) Cancer Res. 70, 10433-10444). These transcription factors are overexpressed in various cancers and are associated with their malignant progression. Collectively, these molecular events cooperate, allowing cancer cells to survive and acquire more aggressive and migratory behaviors during the transition to metastatic and recurrent disease states.

With growing evidence supporting the role of CSCs in tumorigenesis (Sell et al. (2009) Adv. Drug Deliv. Rev. 61, 1007-19), tumor heterogeneity (Gurski et al. (2009) Biomaterials 30, 6076-85), resistance to chemotherapeutic and radiation therapies (Ulrich et al. (2010) Biomaterials 31, 1875-84; Lin & Chang (2008) Biotechnol. J. 3, 1172-84), and the metastatic phenotype (Li et al. (2011) J. Biomol. Screen. 16, 141-54), the development of specific therapies that target CSCs holds promise for improving survival and quality of life for cancer patients, especially those with metastatic disease (Karlsson et al. (2012) Exp. Cell Res. 318, 1577-85; Dhiman et al. (2005) Biomaterials 26, 979-86). Major barriers in studying these cells, however, include their low abundance in vivo (<1%) and the phenotypic plasticity they exhibit during expansion.

Thus, there is an urgent need for the development of novel methods for the expansion of CSCs that can then be used, for example, in investigations of CSC drug targets, CSC-related biomarkers, and CSC pathways that affect fundamental processes associated with carcinogenesis, tumor progression, maintenance, recurrence, and metastasis. The ablation of CSCs may provide a much coveted ‘cure’ for cancers and the search for agents that can specifically target these molecular events associated with CSCs is under intense investigation. However, the presence of only a small percentage of these cells in tumors, makes it difficult to isolate such cells. Novel approaches to expand CSCs in vitro remain an urgent unmet need.

### BRIEF DESCRIPTION OF THE FIGURES

FIG. 1 MCF-7 monolayer cells with different percent of CSCs (A) and (B) were used to grow first-generation tumoroids. Monolayer cells were plated on a scaffold (here, the FiSS^{CSC} platform; Girard et al. (2013) PLoS ONE 8, e75345) for 6 days and the resulting first-generation tumoroids were visualized using NucBlue®. The MCF-7 tumoroids were then processed for single-cell suspensions and stained with CD44 and CD24 fluorochrome antibodies. The CD44^{high} CD24^{low} cells were then detected using flow cytometry and analyzed using the FlowJo software.

FIG. 2 MCF-7 cells were plated on a scaffold (here, FiSS^{CSC} platform) for 6 days to generate first-generation tumoroids (scaffold, first generation). The first-generation tumoroids were then processed and re-plated on the FiSS^{CSC} platform and allowed to grow into second-generation tumoroids (scaffold, second generation). The first- and second-generation tumoroids were visualized using NucBlue®. The MCF-7 tumoroids where then processed for single cell suspensions and stained with CD44 and CD24 fluorochrome antibodies. The CD44^{high} CD24^{low} cells were detected using flow cytometry and analyzed using the FlowJo software.

FIG. 3 MCF-7 monolayer cells were plated on the FiSS^{CSC} platform using regular medium (scaffold) and regular medium supplemented with 50 μM cobalt chloride (scaffold+CoCl₂). After 6 days, the developed tumoroids were visualized using NucBlue®. The MCF-7 tumoroids were then processed for single cell suspensions and stained with CD44 and CD24 fluorochrome antibodies. The CD44^{high} CD24^{low} cells were detected using flow cytometry and analyzed using the FlowJo software.

FIG. 4 MCF-7 monolayer cells where plated on the FiSS^{CSC} platform (scaffold) or FiSS^{CSC} that was manipulated to contain 100 μM cobalt chloride (CoCl₂ scaffold). After 6 days, the developed tumoroids were visualized using NucBlue®. The MCF-7 tumoroids where then processed for single cell suspensions and stained with CD44 and CD24 fluorochrome antibodies. The CD44⁺ CD24⁻ cells were detected using flow cytometry and analyzed using the FlowJo software.

FIG. 5 Second-generation tumoroids showed upregulation of transcription factors that regulate stemness. MCF-7 cells were seeded on FiSS^{CSC} for 6 days to form first-generation tumoroids. These were harvested and cultured to form second-generation tumoroids on FiSS^{CSC} for another 6 days. At the end of each culture period, tumoroids were processed for RNA extraction and subjected to qRT-PCR using probes for Sox-2, Oct-4, and Nanog. HPRT was used as a housekeeping gene control and to normalize gene expression. Data are expressed as means±SEMs. Assays were performed in quadruplicate (* p<0.05).

FIG. 6 CSC populations were maintained when scaling up from 96-well to 6-well FiSS^{CSC} plates. MCF-7 cells were seeded at different cell numbers on FiSS^{CSC} for 6 days to form tumoroids in 6-well plates. Cells plated on monolayers and 96-well FiSS^{CSC} plates were used as controls. At the end of 6 days, the cells were stained with NucBlue® and the live tumoroids were visualized and imaged using fluorescence microscopy (A). Additionally, cells were processed into single cell suspensions and stained with CD44-FITC and CD24-APC antibodies and analyzed using flow cytometry (B).

FIG. 7 The CSC population was potentiated when tumoroids were cultured in CAF CM. MCF-7 cells were seeded on FiSS^{CSC} for 6 days to form tumoroids. Cells were exposed to different concentrations of CAF CM and tumoroids grown on regular medium (RM) were used as controls. At the end of 6 days, the cells were stained with NucBlue® and live tumoroids were visualized and imaged using fluorescence microscopy (A). Additionally, cells were processed into single cell suspensions, stained with CD44-FITC and CD24-APC antibodies, and analyzed using flow cytometry (B). The fold-change in the CD44⁺ CD24⁻ population was plotted for the different conditions.

FIG. 8 The CSC population was potentiated in MCF-7-MCTs containing MDSCs. MCF-7 cells were co-cultured with human MDSCs on FiSS^{CSC} for 6 days to form MCTs. Single-cell tumoroids (SCTs) grown on regular medium (scaffold) were used as a control. At the end of 6 days, the cells were stained with NucBlue® and live tumoroids were visualized and imaged using fluorescence microscopy (A). Additionally, cells were processed into single-cell suspensions, stained with CD44-FITC and CD24-APC antibodies, and analyzed using flow cytometry (B). The percentage of the CD44⁺ CD24⁻ population was plotted for the different conditions.

FIG. 9 CSC expansion in LLC1 cells and tumors cultured on FiSS. (A) Aldefluor assay of LLC1 cells cultured for 6 days either on monolayer or on a FiSS. The baseline fluorescence was established by inhibiting ALDH activity with diethyl amino-benzaldehyde (DEAB). First generation tumoroids were trypsinized and replated on FiSS for additional 6 days to derive second- and then third-generation tumoroids. (B) ALDH+LLC were collected from scaffolds using fluorescence activated cell sorting (FACS). Parental LLC1 or ALDH+LLC1 (sorted) were injected into the flanks of C57BL/6 mice and tumor growth was measured. (C) ALDH-positive populations in LLC1 tumors (left) (10%) vs. in 6-day post culture on FISS (right) (55%), determined by flow cytometry.

FIG. 10 (A) CD44^{high}CD24^{low} populations in A549 xenografts (left) vs. in 6-day post culture on FISS (right), determined by flow cytometry. (B) Sorted CD24 depleted cells were injected subcutaneously into NSG mice and tumor growth was monitored over 60 days. Mice were euthanized when tumors reached 150 mm³.

FIG. 11 Storage of purified cancer stem cells. MACS enrichment of A549 CD44⁺ CD24⁻ cells was analyzed by flow cytometry and are shown Pre-enrichment (A) and post-enrichment (B). C) A549 parental cell line cultured on scaffold (26% CD44⁺ CD24⁻), and D) A549 CD24 depleted by MACS then frozen and thawed to grow as a monolayer (55.9% CD44⁺ CD24⁻).

### DETAILED DESCRIPTION OF THE INVENTION

In one aspect, the invention provides a method for expanding cancer stem cells (CSCs) comprising the steps of: growing tumoroids on a three-dimensional scaffold in an in vitro cell culture; and, isolating CSCs from the tumoroids.

Depending on the cancer cell type and the size of the cell culture, e.g. a 96-well cell culture dish or 6-well cell culture plate, the number of cells seeded typically range between 5-10,000 cells, more preferably 3,000-6,000 cells per well/dish. However, single cells can be plated as well as tumor fragments. The tumoroids generally range in size from 10-1000 microns, more preferably 25-700 microns, and even more preferably 50-300 microns.

In one embodiment, said method further comprises the step of cell dissociation of said tumoroids. In a further embodiment, cell dissociation is performed using a composition comprising an enzyme(s) with proteolytic activity, e.g., ACCUTASE® or trypsin/EDTA. The tumoroids may be dissociated into single cells or tumoroid cell fragments of less than 1,000, 500, 100, 50, or 10 cells. Tumoroid dissociation typically comprises forming a single-cell suspension of tumoroid cancer cells from said tumoroids. Isolation of CSCs from said tumoroids comprise forming a single-cell suspension of tumoroid cancer cells from the tumoroids and isolating CSCs from the single-cell suspension.

In one embodiment, the invention provides for a method of expanding cancer stem cells (CSCs) comprising the steps of:
a) growing a first population of cancer cells in an in vitro cell culture comprising a three-dimensional scaffold;
b) growing tumoroids from said first population of cancer cells on said scaffold;
c) harvesting said tumoroids from said cell culture comprising cell dissociation of said tumoroids into a second population of cancer cells (tumoroid cancer cells); and,
d) isolating CSCs from said tumoroids.

In another embodiment, the invention provides for a method of expanding cancer stem cells (CSCs) comprising the steps of:
a) growing cancer cells in an in vitro cell culture comprising a three-dimensional scaffold;
b) growing tumoroids from said cancer cells on said scaffold;
c) harvesting cancer cells from said tumoroids (tumoroid cancer cells);
d) transferring said tumoroid cancer cells to a new in vitro cell culture comprising a three-dimensional scaffold;
e) growing a subsequent generation of tumoroids from said tumoroid cancer cells on said scaffold of said new in vitro cell culture.

In one embodiment, steps c) through e) of the method immediately above are repeated at least once. In further embodiments, steps c) through e) of the method immediately above are repeated at least twice, at least three times, at least four times, at least five times, at least six times, or at least seven times. In a further embodiment, the method comprises the step of isolating CSCs from said tumoroids.

In one embodiment, said first population of cancer cells in the above methods are human cancer cells. In a further embodiment, said human cancer cells are from a human biopsy. In one embodiment, said human cancer cells is selected from the group consisting of astrocytoma, adrenocortical carcinoma, appendix cancer, basal cell carcinoma, bile duct cancer, bladder cancer, bone cancer, brain cancer, brain stem glioma, breast cancer, cervical cancer, colon cancer, colorectal cancer, cutaneous T-cell lymphoma, ductal cancer, endometrial cancer, ependymoma, Ewing sarcoma, esophageal cancer, eye cancer, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer, and Wilms tumor. In a further embodiment, said human cancer cells is selected from the group consisting of breast, colon, head and neck, gastric, lung, brain, endometrial, liver, skin, prostrate, pancreas, ovary, uterus, kidney, and thyroid cancer cells. In another embodiment, said human biopsy is selected from the group consisting of: astrocytoma, adrenocortical carcinoma, appendix cancer, basal cell carcinoma, bile duct cancer, bladder cancer, bone cancer, brain cancer, brain stem glioma, breast cancer, cervical cancer, colon cancer, colorectal cancer, cutaneous T-cell lymphoma, ductal cancer, endometrial cancer, ependymoma, Ewing sarcoma, esophageal cancer, eye cancer, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer and Wilms tumor. In a further embodiment, said human biopsy is a cancer biopsy selected from the group consisting of: a breast, colon, head and neck, gastric, lung, brain, endometrial, liver, skin, prostrate, pancreas, ovary, uterus, kidney, and thyroid cancer biopsy.

The scaffolds of the present invention are three-dimensional scaffolds and typically comprise randomly oriented fibers. In one embodiment, the scaffold fibers are a mixture of poly(lactic-co-glycolic acid) (PLGA) and a block copolymer of polylactic acid (PLA) and monomethoxypolyethylene glycol (mPEG). In a further embodiment, the scaffold is an electrospun mixture of poly(lactic-co-glycolic acid) (PLGA) and a block copolymer of polylactic acid (PLA) and monomethoxypolyethylene glycol (mPEG). Methods of electrospinning PLGA-mPEG-PLA scaffolds are known in the art and described, e.g., in U.S. Pat. No. 9,624,473, incorporated by reference in its entirety. In one embodiment, the scaffold is chitosan coated.

In some embodiments, the ratio of mPEG-PLA to PLGA in each scaffold fiber is approximately 1:4. In other embodiments, the ratio of mPEG-PLA to PLGA in each scaffold fiber is approximately 1:10. In still other embodiments, the ratio of mPEG-PLA to PLGA in each scaffold fiber is approximately 1:2, 1:3, 1:4, 1:5, 1:6, 1:7, 1:8, 1:9, 1:10 or 1:20, or between any two of the previous ratios, e.g., 1:2-1:6.

In one embodiment, the PLGA contains approximately 85% lactic acid and 15% glycolic acid. Also included herein are embodiments, where the lactic acid:glycolic ratio of PLGA is approximately 75:25, 80:20, 85:15, 90:10, or 95:5, or between any two of the previous ratios, e.g., 80:20-90:10.

The mPEG-PLA and PLGA can be formed into fibers via any method known to those of skill in the art. In some embodiments, solutions of mPEG-PLA and PLGA are electrospun to form mPEG-PLA-PLGA fibers. The scaffold fibers can be electrospun at any voltage, flow rate, and distance that provide for a fiber diameter between approximately 0.1-10 microns, 0.1-7 microns, 0.3 and 10 microns, 0.3-6 microns, or more preferably a fiber diameter between approximately 0.69 to 4.18 microns. In one embodiment, solutions of PEG-PLA and PLGA are electrospun at a positive voltage of 16 kV at a flow rate of 0.2 ml/hour and a distance of 13 cm using a high voltage power supply. The fibers are collected onto an aluminum covered copper plate at a fixed distance of approximately 70 mm. The present invention further includes a mPEG-PLA-PLGA scaffold prepared by collecting the electrospun fibers at a fixed distance between approximately 60 mm and 80 mm.

The resulting mPEG-PLA-PLGA scaffold is a three-dimensional fibrous scaffold having pores. In some embodiments, the scaffold comprises pores having a diameter of less than approximately 20 microns. In other embodiments, the scaffold comprises pores having a diameter of less than approximately 50, 25, 15, 10, or 5 microns.

In one embodiment of the present invention, regular growth medium is used to grow one or more generations of the tumoroids. As used herein, “regular growth medium (or media)” means a media that does not contain any exogenous growth factor supplements added to specifically stimulate stem cells.

In one embodiment, said tumoroids are first-generation tumoroids, i.e., tumoroids produced from a source of cancer cells, e.g., cell line, biopsy, other than tumoroids. In one embodiment, the cancer cell line is selected from the group consisting of: MCF-7 cells, MDA-MB cells, MCF-10A breast cancer cells, PC3 prostate cancer cells, B16 melanoma cells, BG-1 ovarian cells, and LLC Lewis lung cancer cells.

First-generation tumoroids can be dissociated into tumoroid cancer cells and used to grow, subsequent, i.e., second-generation tumoroids. Second-generation tumoroids can be dissociated into tumoroid cancer cells and used to grow third-generation tumoroids. The process can be repeated to produce subsequent generations of tumoroids.

At the end of each tumoroid generation, the resulting tumoroid cancer cells may be processed and analyzed to determine whether stem cell markers (e.g., CD44^{+/high} and CD24^{-/low}) are present/absent or high/low and/or to isolate CSCs from non-CSCs. This can be done by routine methods, such as, flow cytometry or magnetic beads. The isolated tumoroid CSCs can be used to grow subsequent generations of tumoroids. For example, tumoroid CSCs can be isolated from one or more, or each generation and used to grow the next generation of tumoroids.

In one embodiment, the first-generation tumoroids have at least a 2-fold, 2.5-fold or 3-fold increase in CSCs, compared to the cancer cells used to grow the first-generation tumoroids. In another embodiment, the second-generation tumoroids have at least a 5-fold, 6-fold, 7-fold, 8-fold, 9-fold, or 10-fold increase in CSCs, compared to the first-generation tumoroids used to grow the second-generation tumoroids. In another embodiment, the second-generation tumoroids have at least a 5-fold, 10-fold, 15-fold, 20-fold, 25-fold, 30-fold, 40-fold, 50-fold, 60-fold, 70-fold, 75-fold, or 80-fold increase in CSCs, compared to the cancer cells used to grow the first-generation tumoroids. In another embodiment, the third-generation tumoroids have at least a 5-fold, 10-fold, 15-fold, 20-fold, 25-fold, 30-fold, 40-fold, 50-fold, 60-fold, 70-fold, 75-fold, or 80-fold increase in CSCs, compared to the cancer cells used to grow the first-generation tumoroids, compared to the first-generation tumoroids, or to the second-generation tumoroids.

In another embodiment, one or more generations of tumoroids are grown in hypoxic conditions or grown in conditions that mimic hypoxic conditions. In a further embodiment, the hypoxic conditions are throughout the culture medium. In one embodiment, the scaffold induces the hypoxic conditions. In a further embodiment, the hypoxic conditions are local to the scaffold. In another embodiment, the scaffold induces the local hypoxic condition. In one embodiment, the growth medium, e.g., regular growth medium, is supplemented with cobalt chloride to mimic hypoxia in the tumoroids. Alternatively, cobalt chloride is infused into the scaffold matrix to ensure sustained hypoxic conditions for tumoroids growing on the scaffold. In one embodiment, the CoCl₂ is added to the mix of said poly(lactic-co-glycolic acid) (PLGA) and a block copolymer of polylactic acid (PLA) and monomethoxypolyethylene glycol (mPEG) prior to electrospinning. In one embodiment, the CoCl₂ is added to the grown medium or scaffold matrix used to grow the first-generation tumoroids. In another embodiment, the CoCl₂ is added to the growth medium or scaffold matrix used to grow one or more, successive generations of tumoroids, e.g., second-generation, third-generation, and/or fourth-generation tumoroids.

In a further embodiment, tumoroids, e.g., first-generation, second generation, third generation, or fourth generation tumoroids, etc., or CSCs isolated from tumoroids, are cultured in conditioned media (CM) collected from primary cancer-associated fibroblasts (CAFs), e.g., CAFs from breast cancer tumors, and/or myeloid-derived suppressor cells (MDSCs) from human peripheral blood, to increase the number of CSCs. In one embodiment, the CAFs are human CAFs.

In another embodiment, tumoroid cultures are expanded from a smaller to larger cell culture format, e.g., from a 96-well format to a six-well format tissue culture dish, to increase the yield of CSCs (by 30-fold), while maintaining the ability for CSC expansion.

In one embodiment, the tumoroids are cultured in a medium comprising an ECM-based hydrogel. In another embodiment, the scaffold comprises an electrospun mixture of poly(lactic-co-glycolic acid) (PLGA) and a block copolymer of polylactic acid (PLA) and monomethoxypolyethylene glycol (mPEG) and the medium comprises an ECM-based hydrogel. In a further embodiment, the ECM-based hydrogel is a solubilized basement membrane preparation extracted from the Engelbreth-Holm-Swarm (EHS) mouse sarcoma, e.g., MATRIGEL®.

In a further embodiment, the method provides for growing a plurality of generations of tumoroids, wherein each generation in succession has a greater percentage of CSCs than the preceding generation of tumoroids, or in the case of the first generation of tumoroids, has a greater percentage of CSCs than the initial culture of cancer cells that gave rise to the first-generation of tumoroids.

In one embodiment, dissociated first-generation tumoroids, e.g., a single-cell suspension of first-generation tumoroid cancer cells, are cultured to grow a second-generation of tumoroids. In a further embodiment, dissociated second-generation tumoroids, e.g., a single-cell suspension of second-generation tumoroid cancer cells, are cultured to grow a third-generation of tumoroids. In a further embodiment, dissociated third-generation tumoroids, e.g., a single-cell suspension of third-generation tumoroid cancer cells, are cultured to grow a fourth-generation of tumoroids. In further embodiments, the process is repeated for a fifth-, sixth-, seventh-, eighth-, ninth-, tenth, or more generations of tumoroids. In one embodiment, a single-cell suspension of tumoroid cancer cells from each generation of tumoroids is used to grow the next generation of tumoroids. In each case the tumoroids are grown in an in vitro cell culture comprising a three-dimensional scaffold according to the invention.

In one embodiment, said CSCs isolated according to the method of the present invention are from: first-generation tumoroids, second-generation tumoroids, third-generation tumoroids, fourth-generation tumoroids. In other embodiments, said CSCs isolated according to the method of the present invention are from the fifth-, sixth-, seventh-, eighth-, ninth-, tenth, or more generations of tumoroids.

In one embodiment, CSCs are isolated from a first-generation of tumoroids and are cultured to grow a second-generation of tumoroids. In a further embodiment, CSCs are isolated from a second-generation of tumoroids and are cultured to grow a third-generation of tumoroids. In a further embodiment, CSCs are isolated from a third-generation of tumoroids and are cultured to grow a fourth-generation of tumoroids. In one embodiment, CSCs isolated from each generation of tumoroids is used to grow the immediate subsequent generation of tumoroids.

In one embodiment, the last generation of tumoroids are harvested. In a further embodiment, CSCs are isolated from the last generation of tumoroids. In a further embodiment, the CSCs isolated from the last generation of tumoroids are: a) grown to the expand the population in an in vitro culture; b) used in an in vitro cell assay, e.g., an assay screening drug compounds, such as anti-cancer drug compounds; c) stored, e.g., frozen; or, d) used in an in vivo animal model, e.g., tumor or tumor xenograft model. In one embodiment the animal is a rodent, e.g., mouse (NOD-EGFP mouse) or rat. In a further embodiment, the mouse is a NOD-EGFP mouse.

In another embodiment, the culture comprises one or more iron chelators. In a further embodiment, the scaffold further comprises one or more iron chelators. In a further embodiment, said one or more iron chelators is added to a mix of said poly(lactic-co-glycolic acid) (PLGA) and a block copolymer of polylactic acid (PLA) and monomethoxypolyethylene glycol (mPEG) prior to electrospinning.

In another embodiment, said culture or said scaffold comprises a siRNA that knocks down the von Hippel-Lindau (VHL) tumor suppressor gene.

In another embodiment, said culture, scaffold, or cancer cells comprise a heterologous DNA encoding growth factors. In another embodiment, said culture or said scaffold comprises TGF-β.

In one embodiment, all method steps are carried out in vitro. In another embodiment, the method comprises one or more in vivo steps. In one embodiment, cancer cells are injected into a non-human host animal, e.g., rodent such as mouse or rat, to form a tumor (e.g., tumor xenograft). In one embodiment, the host animal is a NOD-EGFP mouse. In one embodiment, the cancer cells injected into a non-human host animal are injected with an ECM-based hydrogel. In a further embodiment, the said ECM-based hydrogel is a solubilized basement membrane preparation extracted from the Engelbreth-Holm-Swarm (EHS) mouse sarcoma (e.g., MATRIGEL®).

In a one embodiment, the tumor is removed from the host animal. The tumor is dissociated into a suspension of tumor cells or tumor fragments and are cultured in vitro on a scaffold to grow tumoroids according to the method of the present invention. In a further embodiment, CSCs are isolated from the tumoroids. In another embodiment, the CSCs isolated from the tumoroids or the tumoroid cancer cells are cultured and grown on a scaffold to produce a subsequent generation of tumoroids. In a further embodiment, CSCs are isolated from the tumor/tumor xenograft cancer cells. In a further embodiment, the CSCs isolated from the tumor/tumor xenograft cancer cells are cultured in vitro on a scaffold to grow tumoroids according to the present invention. In one embodiment, the cancer cells injected into the host animal are tumoroid cells of the present invention, cells from a tumor, e.g., human tumor, or a cancer cell line. In a further embodiment, the tumoroid cells injected into the host animal are first-generation, second-generation, or third-generation tumoroid cells. In a further embodiment, the tumoroid cells injected into the host animal are CSCs isolated from tumoroids.

In another aspect, the invention relates to a method of screening a drug compound, e.g., an anti-cancer compound. In one embodiment, the method comprises: a) culturing the tumoroids of present invention; b) contacting the tumoroids with a drug compound; and c) measuring the effect of the drug compound on the tumoroids. In another embodiment, the method comprises: a) culturing the tumoroid cancer cells of the present invention; and b) contacting the tumoroid cancer cells with the drug compound; and c) measuring the effect of the drug compound on the tumoroid cancer cells. In another embodiment, the method comprises: a) culturing the isolated CSCs of the present invention; and b) contacting the isolated CSCs with the drug compound; and c) measuring the effect of the drug compound on the isolated CSCs.

In one embodiment the method comprises measuring an IC₅₀, GI₅₀, ED₅₀ or LD₅₀. IC₅₀ is the drug concentration resulting in 50% inhibition of a desired activity. GI₅₀ is the concentration for 50% of maximal inhibition of cell proliferation. GI₅₀ is preferably used for cytostatic (as opposed to cytotoxic) agents. ED₅₀ (or EC₅₀) is the Effective Dose (or Effective Concentration) resulting in 50% of maximum effect for any measured biological effect of interest, including cytotoxicity. Lethal Dose 50 (LD₅₀) is the concentration resulting in 50% cell death.

This invention, in part, relates to expanding cancer stem cell numbers using, for example, the FiSS™ platform, with which we have shown several-fold amplification of CSC numbers using the MCF7 breast cancer cell line, as an example. The Table 1 summarizes these findings.

These results showed that several variables affected CSC expansion. Cumulatively, a total of over 80-fold expansion was achieved with MCF cancer stem cells, as an example, by growing them on the FiSS™ platform. Also, biopsy cell xenografts, such as A549 (lung cancer), cultured on FiSS™ showed expanded CSCs in the first generation that were higher than the MCF7 cell-line induced expansion of CSCs on the FiSS™ scaffold.

TABLE 1
CSC Expansion

| Variable | Fold Amplification |
|---|---|
| Cell line (e.g., MCF7) | |
| First-generation scaffold | 3.3 |
| Second-generation scaffold | 10 |
| CoCl₂ in scaffold | 1.3 |
| CAF-conditioned medium | 1.5 |
| MDSC-condition medium | 1.3 |
| Total | 83.6 |
| Tumor biopsy (xenograft) | |
| First-generation scaffold | 8 |

Several factors play a role in CSC expansion on scaffolds, such as FiSS™. These include physical modifications, physiological, biochemical, and biological factors that showed enhanced CSC numbers in organotypic FiSS™ tumoroids. In terms of physical conditions, there are many variations on the FiSS™ scaffold materials and other scaffold materials that can also serve to amplify CSCs. Similarly, among physiological niches, our results showed that hypoxic conditions may promote stemness. Thus, scaffolds that induces hypoxic conditions are valuable, as we showed by introducing CoCl₂ into the scaffold. Scaffolds with DNA encoding growth factors may also increase the stem cell amplification potential. Other ways to generate hypoxia include adding iron chelators, indicating that the stimuli may interact through effects on a ferroprotein oxygen sensor. Furthermore, knocking down the von Hippel-Lindau (VHL) tumor suppressor gene, such as by linking a siRNA to the scaffold may increase HIF 1a and hypoxia-like regulation.

Furthermore, addition of other factors to the cell culture medium for culturing tumoroids can produce the amplification of CSCs. Thus, conditioned media from cancer-associated fibroblast cultures or from cultures of myeloid-derived suppressor cells can enhance CSC expansion. Similarly, tumor infiltrates from patient tumors may also enhance CSC numbers. Adding small amounts of Matrigel® (^{˜}1 to ^{˜}3%) can increase CSC numbers. Moreover, adding growth factors, such as TGF-β and/or SDF1 was found to increase stem cell amplification by up to ^{˜}5-fold. Similarly, other growth factors may also be valuable in amplifying CSC expansion.

The present invention describes methods to increase the population of cancer stem cells (CSCs) using, for example, a FiSS™ (fiber-inspired smart scaffold) platform. In one embodiment, regular growth medium was used to grow first-generation MCF-7 tumoroids, and a protocol was developed to grow second-generation tumoroids from the first-generation MCF-7 tumoroids. At the end of each generation, we processed the resulting tumoroids and analyzed the cells for stem cell markers (e.g., CD44^{high} and CD24^{low}) by flow cytometry. The results showed that the first-generation MCF-7 tumoroids gave a ^{˜}3-fold increase in CSCs.

Embodiments of this invention include a series of methods to expand cancer stem cells (CSCs) using, for example, a polymeric nanofiber scaffold, such as the fiber-inspired smart scaffold (FiSS™) platform, a scaffold for cell culture comprising an electrospun mixture of poly(lactic-co-glycolic acid) (PLGA) and a block copolymer of polylactic acid (PLA) and monomethoxypolyethylene glycol (mPEG), on which the culture of cancer cells results in the formation of tumor-like structures, referred to here as “tumoroids.” More specifically, “tumoroids” are a compact aggregate of cancer cells with or without any other stromal cells cultured on a 3D polymeric scaffold that morphologically, physiologically and biochemically resembles tumors. We found that tumoroids growing on such scaffolds showed markers of EMT, as observed by the upregulation of vimentin and downregulation of E-cadherins. Increases in EMT markers resulted in increases in the population of the CD44⁺ CD24⁻ cells. We also confirmed that the increase in CSC populations correlated with an increase in aldehyde dehydrogenase activity. Embodiments of our invention provide methods for amplifying cancer stem cells (CSCs) from cancer cells. Further embodiments of our invention provide methods for amplifying human cancer stem cells (CSCs) from human cancer cells.

MCF-7 single-cell tumoroids grown in regular medium increased the number of CSCs in the first generation. We grew MCF-7 cells in regular growth medium. The cells were plated on scaffolds in a 96-well cell culture plate. Fresh medium was added on the second day post-seeding and on day 6 post-seeding, the tumoroids were visualized. After confirming the presence of healthy looking tumoroids, they were detached from the scaffold and processed for single cell suspensions using accutase:citrate solution. The single cell suspension was then counted for viability and stained with human anti-CD44-APC-cy7 and anti-CD24-APC antibodies. DAPI was used to differentiate the live cells within the single-cell population and the CD44⁺ CD24⁻ cell population was determined using flow cytometry. MCF-7 cells formed well-developed first-generation SCTs after 6 days on FiSS^{CSC} using regular growth medium. Importantly, regardless of the percentage of the CSC population in the monolayer MCF-7 cells, the first-generation tumoroids consistently showed a 3-fold increase in their CSC population, as determined by the increase in the CD44⁺ CD24⁻ cell population.

Second-generation MCF-7 tumoroids further expanded CSCs in regular medium. We first grew first-generation tumoroids, as described before. The first-generation tumoroids were then processed for single-cell suspensions and plated on scaffolds in a 96-well cell culture plate. Fresh medium was added on the second day post-seeding and on day 6 post-seeding, the second-generation tumoroids were visualized. After confirming healthy looking tumoroids, they were detached and the single-cell suspension was stained with human anti-CD44-APC-cy7 and anti-CD24-APC antibodies. Non-DAPI stained live cells were used to determine the CD44⁺ CD24⁻ cell population using flow cytometry. The first-generation tumoroids gave a ^{˜}3-fold increase in CSCs, which was increased exponentially, by ^{˜}10-fold, in the second-generation MCF-7 tumoroids.

We characterized CSCs within MCF-SCTs grown under hypoxic conditions. Because hypoxia has been suggested to be required for the maintenance of CSCs, we tested this in our 3D model using cobalt chloride (CoCl₂), a known inducer of hypoxia. For this, we plated MCF-7 cells in regular growth medium supplemented with 50 μM cobalt chloride. As before, the tumoroids where visualized on day 6 post-seeding and then processed for flow cytometry using human anti-CD44-APC-cy7 and anti-CD24-APC antibodies. DAPI was used to differentiate the live cells within the single-cell population and the CD44⁺ CD24⁻ cell population was determined using flow cytometry. The results showed that the addition of cobalt chloride did not change the percentage of CSCs in the first-generation MCF-7 tumoroids markedly. This absence of the amplification of CSCs may be attributable to the inability of externally added cobalt chloride to maintain hypoxic conditions throughout the duration of the cell culture. Frequent replenishment of cobalt chloride may be necessary to ensure sustained hypoxia.

We characterized CSCs in first-generation MCF-7-SCTs grown on scaffolds containing cobalt chloride. Because our earlier experiment showed the inability of externally added cobalt chloride to increase CSCs in first-generation tumoroids markedly, we incorporated cobalt chloride within the matrix of the scaffold. Our assumption was that cobalt chloride embedded within the scaffold would aid in maintaining a hypoxic cell culture environment throughout the duration of experiment. For this purpose, we mixed 100 μM cobalt chloride with a mix of polymers and the resulting electrospun scaffold was used to test its effect on the growth of MCF-7-SCTs. We plated MCF-7 cells in regular growth medium on our cobalt chloride-containing scaffold. As before, the tumoroids where visualized on day 6 post-seeding and before conducting flow cytometry, we first determined the ability of the cobalt chloride within the scaffold to maintain hypoxic conditions. Hypoxic regions in the MCF-7 SCTs were detected using fluorogenic probes for hypoxia, which take advantage of the nitroreductase activity present in hypoxic cells by converting the nitro group to hydroxylamine (NHOH) and amino (NH₂) and releasing the fluorescent probe. After 6 days in culture, only MCF-7 SCTs grown on the cobalt chloride scaffold, but not the regular scaffold, showed fluorescence, demonstrating the ability of the cobalt chloride-containing scaffold to maintain hypoxia. We then processed the cells for flow cytometry using human anti-CD44-APC-cy7 and anti-CD24-APC antibodies. DAPI was used to differentiate the live cells within the single-cell population and the CD44⁺ CD24⁻ cell population was determined using flow cytometry. MCF-7 SCTs showed an increase in the CSC population, which was slightly higher than that observed in first-generation MCF-7 SCTs grown on regular scaffolds.

The increased CD44⁺ CD24⁻ MCF-7 cell population correlated with upregulation of transcription factors known to regulate stemness. We previously showed that the CSC population, defined as CD44⁺ CD24⁻ cells, increased progressively in tumoroids when cultured sequentially through first and second generations. Because several markers of stemness have been reported, we sought to ascertain whether the FiSS^{CSC} platform showed an increase in CSCs depending on the markers used. We examined the family of transcription factors, Oct-4, Sox-2, and Nanog, part of the so-called Yamanaka transcription factors. Oct-4, Sox-2, and Nanog are three transcription factors that play important roles in maintaining the pluripotency and self-renewal characteristics of CSCs. As described previously, we cultured the first-generation MCF-7 tumoroids on FiSS^{CSC} for 6 days, after which they were harvested and divided into two groups. One group was subjected to RNA extraction and the second group was further cultured on FiSS^{CSC} to form second-generation tumoroids. At the end of 6 days, the second-generation tumoroids were harvested and subjected to RNA extraction. Extracted RNAs from monolayers and second-generation tumoroids were processed and subjected to qRT-PCR using probes for Oct-4, Sox-2, and Nanog. The results showed that Oct-4, Sox-2, and Nanog were statistically significantly increased in their expression in the second generation versus monolayer cells. Whereas Sox-2 showed a relatively modest increase, Oct-4 and Nanog showed ^{˜}3-4-fold increases in their transcripts, relative to the monolayer cells. This demonstrated that the CSC increase, as demonstrated by the increase in the CD44⁺ CD24⁻ population, correlated with increased gene expression of Oct-4, Sox-2, and Nanog.

The increased CD44⁺ CD24⁻ MCF-7 cell population was maintained when tumoroids were cultured in a 6-well FiSS^{CSC} format. Embodiments of the present invention are useful for increasing the yield of CSCs. We characterized the conditions for growing tumoroids on a 6-well FiSS^{CSC} plate. This upscaling led to a ^{˜}30-fold increase in cell seeding and a consequent increase in processed CSCs at the end of the experiment versus a 96-well plate. Thus, by culturing increased numbers of cells, seeded in, for example, a 6-well plate, it was found that all tested cell numbers gave well-formed tumoroids at the end of day 6. When we examined the CD44⁺ CD24⁻ cell population, we found an increase in CSC numbers. The viability of the cells was comparable to that obtained in the 96-well format.

Exposure of MCF-7 cells to CAF CM increased the population of CSCs in tumoroids cultured on FiSS^{CSC}. It has been reported that CSC maintenance requires steady cues from cellular and non-cellular components present within the tumor microenvironment. Within this phenomenon, the players shown to have roles include CAFs. To assess whether secretory factors from CAFs could expand the population of CSCs, we collected and cultured CAFs from breast cancer patients. Specifically, we cultured CAFs to 80% confluence and then incubated them in growth medium for 48 h, at the end which the medium was collected, centrifuged, and stored at −80° C. until used. Before use, the medium was thawed on ice and appropriate dilutions were made in MCF-7 growth medium for testing. CAF CM at all percentages tested aided the formation of tumoroids on FiSS^{CSC}. Importantly, 10 and 25% CAF CM increased the CSC populations more than was observed with regular growth medium. This confirmed that secretory factors present within CAF CM increased the population of the CD44⁺ CD24⁻ CSCs in MCF-7 tumoroids cultured with the FiSS^{CSC} platform.

We characterized the CSCs in MCF-7 multi-cellular tumoroids (MCTs) grown in co-culture with MDSCs. Cell-cell interaction, especially between cancer cells and immune cells, like MDSCs, has been shown to encourage the induction and maintenance of CSCs in vivo. To test the effects of co-culture, we procured normal healthy whole blood, which was processed for mononuclear cells. Mononuclear cells were then cultured with HeLa cells to help the differentiation of the mononuclear cells to MDSCs. After 6 days in culture, CD33⁺ cells were isolated and characterized for MDSC cell-surface markers using flow cytometry.

MDSCs isolated from three different individuals were co-cultured with MCF-7 cells on a scaffold. The co-culture formed irregular tumoroids that were slightly larger in size than with MCF-SCTs and the CD44⁺ CD24⁻ stem cell-like population showed a slight increase versus MCF-SCTs.

We characterized CSCs in xenografts derived from A549 lung cancer grown on FiSS^{CSC}. We next examined the potential to isolate and expand the rare CSC population from in vivo tumors. To establish xenografts, A549 cells were mixed with Matrigel® and injected into the flanks of female NOD-EGFP mice subcutaneously and we monitored tumor growth. Matrigel® is a proprietary solubilized basement membrane, preparation extracted from the Engelbreth-Holm-Swarm (EHS) mouse sarcoma, a tumor rich in ECM proteins, such as laminin (a major component), collagen IV, heparin sulfate proteoglycans, entactin/nidogen, and some growth factors. Tumors were resected and single-cell suspensions of these tumors were cultured on FiSS^{CSC} for 7 days. A 5-9-fold increase in the CD44⁺ CD24⁻ population, indicating stem-like cells, was found in tumoroids derived from A549 xenograft cells cultured on FiSS^{CSC} versus A549 xenografts. Moreover, the injection of CD44⁺ CD24⁻ cells in NSG mice could initiate tumors in vivo, suggesting that CD44⁺ CD24⁻ cells truly represent CSC-like cells in A549.

We obtained ^{˜}10⁶ cells per A549 xenograft and after culturing on one 6-well format FiSS^{CSC} plate; we enriched by ^{˜}50% the cells expressing CD44⁺ CD24⁻. Thus, it is possible to collect ^{˜}10⁷ CD44⁺ CD24⁻ cells from ^{˜}20 A549 xenografts. Similar strategies can be used isolate CD44⁺ CD24⁻ CSC-like cells from other xenografts grown with different cell types, including human cells.

In summary, the present invention describes methods to increase the population of cancer stem cells (CSCs) using, for example, a FiSS™ (fiber-inspired smart scaffold) platform. Indeed, the present invention describes a protocol for the large-scale enrichment of cancer stem cells (CSCs) using a scaffold, such as the fiber-inspired smart scaffold (FiSS^{CSC}). We used different cell culture conditions, such as growing the cells under hypoxic conditions, using conditioned media (CM) from tumor stromal cells, co-culturing cancer cells with stromal cells, and using exogenous soluble factors.

Our findings showed that MCF-7 breast cancer cells formed tumoroids on the FiSS^{CSC}. These tumoroids harbored ^{˜}3-5-fold more CD44⁺ CD24⁻ stem-like cells versus cells grown as a monolayer. Moreover, we correlated the increase in the CD44⁺ CD24⁻ stem-like cells in the tumoroids with increased expression of Sox-2, Oct-4, and Nanog, which are known to confer stemness in cells. The MCF-7 CD44⁺ CD24⁻ stem-like cell population did not increase markedly when the tumoroids were grown on a scaffold infused with cobalt chloride to mimic hypoxia. MCF-7 cells formed tumoroids when co-cultured with human immune cells: specifically, MDSCs. The CD44⁺ CD24⁻ stem-like population was comparable to that with single-cell tumoroids of MCF-7 cells. MCF-7 cells formed tumoroids when exposed to CM from human cancer-associated fibroblasts (CAFs).

We scaled-up the protocol for tumoroid formation from, for example, a 96-well format to, for example, a 6-well format. This resulted in a 30-fold increase in cell number input while maintaining the fold increase in the CD44⁺ CD24⁻ stem-cell like population we observed in the 96-well format. We harvested first-generation tumoroids and reseeded them to form second- and third-generation tumoroids. Within each succeeding generation, we found an increase in the CD44⁺ CD24⁻ stem cell-like population. We used microbeads and processed CD44⁺ cells and froze them. Furthermore, we demonstrated that on thawing, these cells grew into tumoroids on the scaffold and maintained the population of CD44⁺ CD24⁻ cells.

### EXAMPLES

### Example 1. MCF-7 Single-Cell Tumoroids Grown in Regular Medium Increased the Number of CSCs in the First Generation

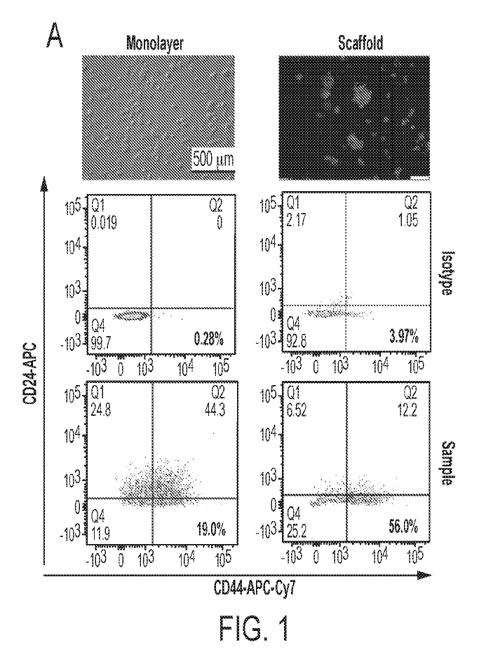

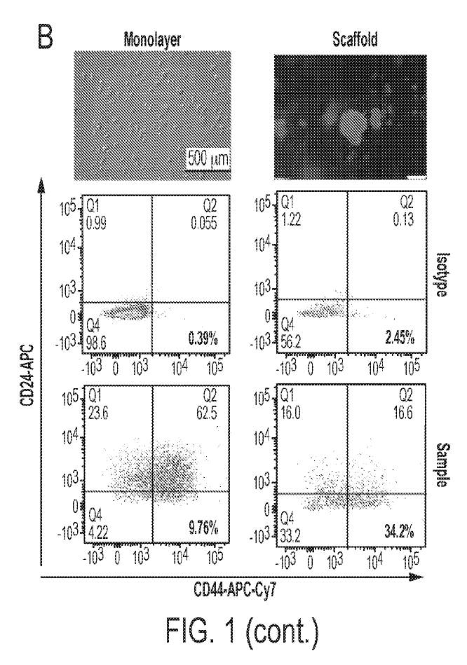

We first plated MCF-7 cells in regular growth medium, consisting of RPMI-1640 supplemented with 10% fetal bovine serum (FBS) and 1× penicillin-streptomycin. The cells were plated at ^{˜}7,000 cells per scaffold in a 96-well cell culture plate. Fresh medium was added on the second day post-seeding and on day 6 post-seeding, the tumoroids were visualized using NucBlue® dye. After confirming the presence of healthy looking tumoroids, they were detached from the scaffold and processed for single cell suspensions using accutase:citrate solution (1:1 ratio). The single cell suspension was then counted for viability and stained with human anti-CD44-APC-cy7 and anti-CD24-APC. DAPI was used to differentiate the live cells within the single cell population and the CD44⁺ CD24⁻ cell population was determined from the live cells using flow cytometry. As seen in FIGS. 1A and 1B, MCF-7 cells formed well-developed first-generation SCTs after 6 days on FiSS^{CSC} using regular growth medium. Importantly, regardless of the percentage of the CSC population in the monolayer MCF-7 cells, the first-generation tumoroids consistently showed a ^{˜}3-fold increase in their CSC population, as determined by the increase in the CD44⁺ CD24⁻ cell population.

### Example 2. Second-Generation MCF-7 Tumoroids Further Expanded CSCs in Regular Medium

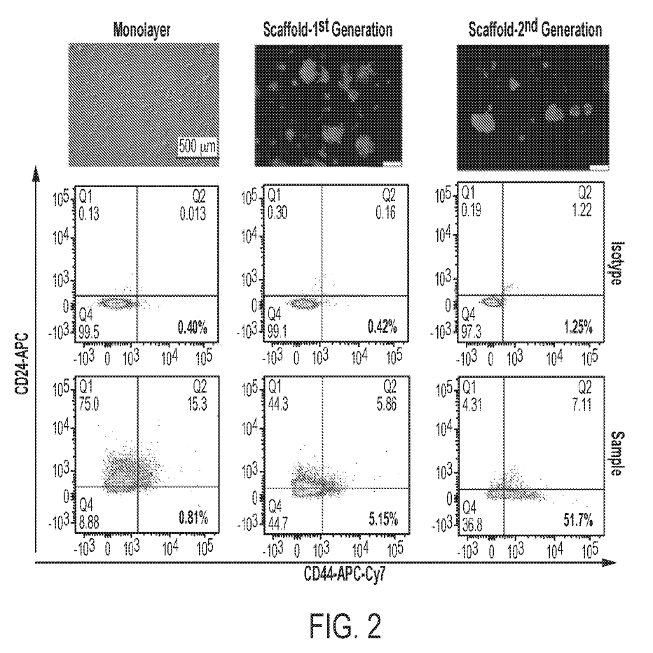

We first grew first-generation tumoroids, as described before. The first-generation tumoroids were then processed for single-cell suspensions and plated at ^{˜}8,000 cells per scaffold in a 96-well cell culture plate. Fresh medium was added on the second day post-seeding and on day 6 post-seeding, the second-generation tumoroids were visualized using NucBlue® dye. After confirming healthy looking tumoroids, they were detached and the single-cell suspension was stained with human anti-CD44-APC-cy7 and anti-CD24-APC. Non-DAPI stained live cells were used to determine the CD44⁺ CD24⁻ cell population using flow cytometry. Thus, the first-generation tumoroids gave an ^{˜}3-fold increase in CSCs, which was increased exponentially, by ^{˜}10-fold, in the second-generation MCF-7 tumoroids (FIG. 2).

### Example 3. Characterization of CSCs in MCF-SCTs Grown in Hypoxic Conditions

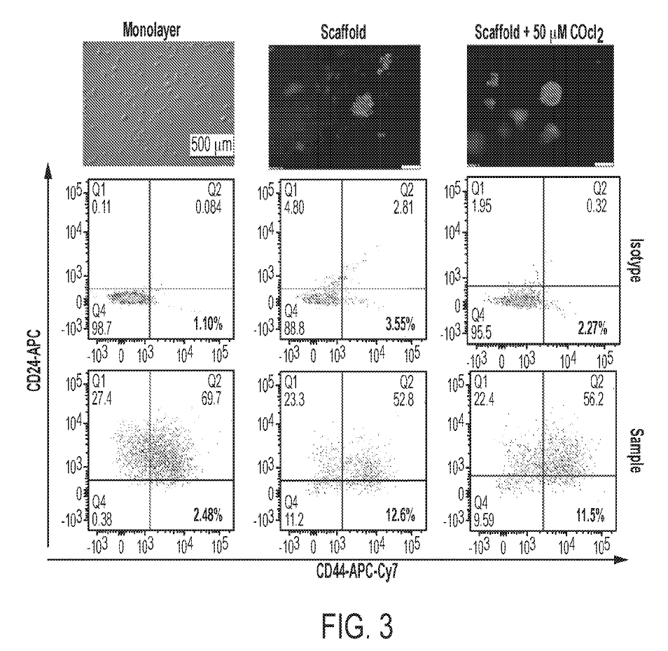

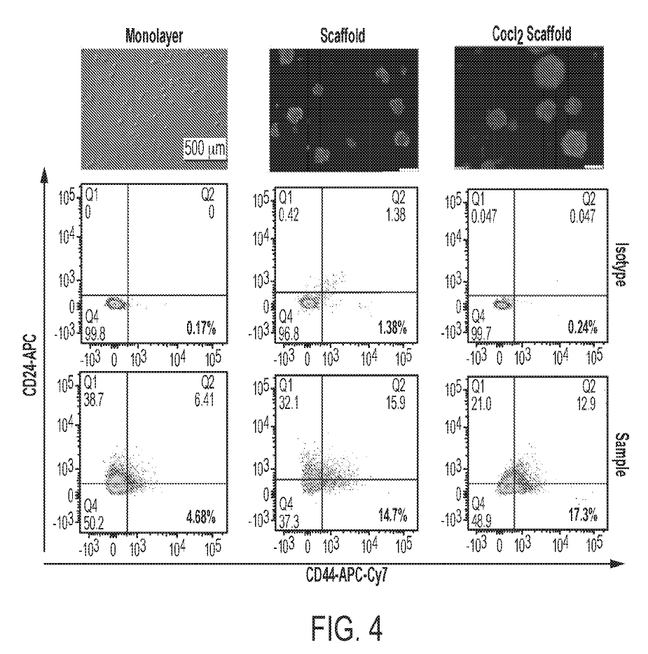

Because hypoxia appears to be required for the maintenance of CSCs, we wanted to test this in our 3D model using cobalt chloride, a known inducer of hypoxia. For this, we plated MCF-7 cells in regular growth medium supplemented with 50 μM cobalt chloride. As before, the tumoroids where visualized on day 6 post-seeding and then processed for flow cytometry using human anti-CD44-APC-cy7 and anti-CD24-APC. DAPI was used to differentiate the live cells within the single-cell population and the CD44⁺ CD24⁻ cell population was determined using flow cytometry. The results showed that the addition of cobalt chloride did not change the percentage of CSCs in the first-generation MCF-7 tumoroids (FIG. 3). This absence of the amplification of CSCs may be attributable to the inability of externally added cobalt chloride to maintain hypoxic conditions throughout the duration of the cell culture. Frequent replenishment of cobalt chloride may be necessary to ensure sustained hypoxia.

### Example 4. Characterization of CSCs in First-Generation MCF-7-SCTs Grown on Scaffolds Containing Cobalt Chloride

Because our earlier experiment showed the inability of externally added cobalt chloride to increase CSCs in first-generation tumoroids, we decided to incorporate cobalt chloride within the matrix of the scaffold. Our assumption was that cobalt chloride embedded within the scaffold would aid in maintaining a hypoxic cell culture environment throughout the duration of experiment. For this purpose, we mixed 100 μM cobalt chloride with a proprietary mix of polymers and the resulting electrospun scaffold was used to test its effect on the growth of MCF-7-SCTs. We plated MCF-7 cells in regular growth medium on our cobalt chloride-containing scaffold. As before, the tumoroids where visualized on day 6 post-seeding using NucBlue® and before conducting flow cytometry, we first determined the ability of the cobalt chloride within the scaffold to maintain hypoxic conditions. Hypoxic regions in the MCF-7 SCTs were detected using fluorogenic probes for hypoxia (red), which take advantage of the nitroreductase activity present in hypoxic cells by converting the nitro group to hydroxylamine (NHOH) and amino (NH₂) and releasing the fluorescent probe. After 6 days in culture, only MCF-7 SCTs grown on the cobalt chloride scaffold, but not the regular scaffold, showed red fluorescence demonstrating the ability of the cobalt chloride-containing scaffold to maintain hypoxia. We then processed the cells for flow cytometry using human anti-CD44-APC-cy7 and anti-CD24-APC. DAPI was used to differentiate the live cells within the single-cell population and the CD44⁺ CD24⁻ cell population was determined using flow cytometry. MCF-7 SCTs showed an increase in the CSC population, which was slightly higher than that observed in first-generation MCF-7 SCTs grown on regular scaffolds (FIG. 3).

### Example 5. The Increased CD44⁺ CD24⁻ MCF-7 Cell Population Correlated with Upregulation of Transcription Factors Known to Regulate Stemness

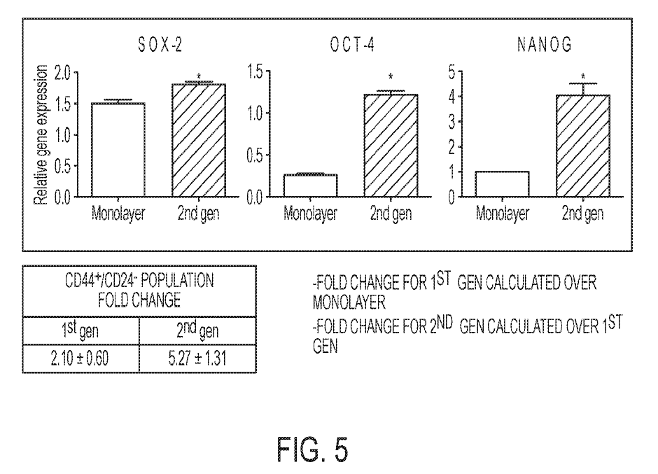

We previously showed that the CSC population, defined as CD44⁺ CD24⁻ cells, increased progressively in tumoroids when cultured sequentially through first and second generations. Because several markers of stemness have been reported, we sought to ascertain whether the FiSS^{CSC} platform showed an increase in CSCs depending on the markers used. We examined the family of transcription factors, Oct-4, Sox-2, and Nanog, that are part of the so-called Yamanaka transcription factors. Oct-4, Sox-2, and Nanog are three basic transcription factors that play important roles in maintaining the pluripotency and self-renewal characteristics of CSCs. As described previously, we cultured the first-generation MCF-7 tumoroids on FiSS^{CSC} for 6 days, after which they were harvested and divided into two groups. One group was subjected to RNA extraction using the Trizol reagent and the second group was further cultured on FiSS^{CSC} to form second-generation tumoroids. At the end of 6 days, the second-generation tumoroids were harvested and subjected to RNA extraction. Extracted RNAs from monolayers and second-generation tumoroids were processed and subjected to qRT-PCR using probes for Oct-4, Sox-2, and Nanog. HPRT was used as a housekeeping gene to normalize gene expression. The results showed that Oct-4, Sox-2, and Nanog showed statistically significant increases in their expression in the second generation when compared with the monolayer (FIG. 5). While Sox-2 showed a relatively modest increase, Oct-4 and Nanog showed ^{˜}3-4-fold increases in their transcripts, relative to the monolayer cells. This demonstrated that the CSC increase, as demonstrated by the increase in the CD44⁺ CD24⁻ population, correlated increased gene expression of Oct-4, Sox-2, and Nanog.

### Example 6. The Increased CD44′CD24⁻ MCF-7 Cell Population was Maintained when Tumoroids were Cultured in a 6-Well FiSS^{CSC} Format

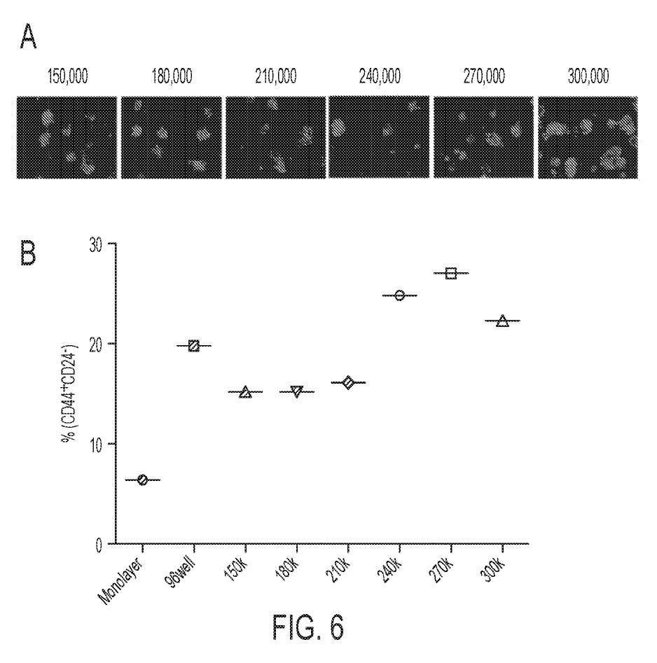

Embodiments of the present invention are useful for increasing the yield of CSCs. We characterized the conditions for growing tumoroids on a 6-well FiSS^{CSC} plate. This upscaling led to a 30-fold increase in cell seeding and a consequent increase in processed CSCs at the end of the experiment versus a 96-well plate. Thus, by culturing increased numbers of cells, seeded in, for example, a 6-well plate, it was found that all tested cell numbers gave well-formed tumoroids at the end of day 6 (FIG. 6A). When we examined the CD44⁺ CD24⁻ cell population, we found an increase in CSC numbers, although this varied slightly from experiment to experiment. Specifically, we found that there was a cell density that gave high numbers of CD44⁺ CD24⁻ cells in the 6-well format versus the 96-well format. This cell number was between ^{˜}240,000 and ^{˜}270,000 cells per/well in a 6-well plate (FIG. 6B). Cell numbers lower or higher than this range resulted in a decreased CD44⁺ CD24⁻ cell population. The viability of the cells was comparable to that obtained in the 96-well format (^{˜}80%). Thus, in one embodiment of the invention, ^{˜}240,000 cells/6 wells were used for growing tumoroids on FiSS^{CSC}.

### Example 7. Exposure of MCF-7 Cells to CAF CM Increased the Population of CSCs in Tumoroids Cultured on FiSS^{CSC}

It has been reported that CSC maintenance requires steady cues from the cellular and non-cellular components present within the tumor microenvironment. Within this phenomenon, the players shown to have roles include CAFs. To assess whether secretory factors from CAFs could expand the population of CSCs, we collected and cultured CAFs from breast cancer patients. Specifically, we cultured CAFs to ^{˜}80% confluence and then incubated them in growth medium for 48 h, at the end which the medium was collected, centrifuged, and stored at −80° C. until used. Before use, the medium was thawed on ice and appropriate dilutions were made in MCF-7 growth medium for testing.

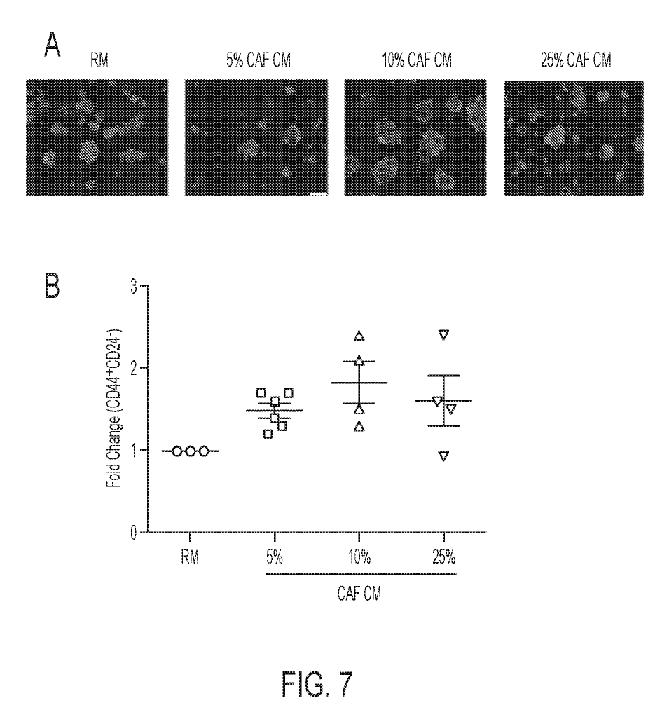

As shown in FIG. 7A, CAF CM at all percentages tested, aided the formation of tumoroids on FiSS^{CSC}. Importantly, 10 and 25% CAF CM increased the CSC populations more than was observed with regular growth medium (FIG. 7B). This confirmed that secretory factors present within CAF CM increased the population of the CD44⁺ CD24⁻ CSCs in MCF-7 tumoroids cultured with the FiSS^{CSC} platform.

### Example 8. Characterization of CSCs in MCF-7 Multi-Cellular Tumoroids (MCTs) Grown in Co-Culture with MDSCs

Cell-cell interaction, especially between cancer cells and immune cells, like MDSCs, has been shown to encourage the induction and maintenance of CSCs in vivo. To test the effects of co-culture, we procured normal healthy whole blood, which was processed for mononuclear cells. Mononuclear cells were then cultured with HeLa cells (1:100 ratio) to help the differentiation of the mononuclear cells to MDSCs. After 6 days in culture, CD33⁺ cells were isolated and characterized for MDSC cell-surface markers using flow cytometry.

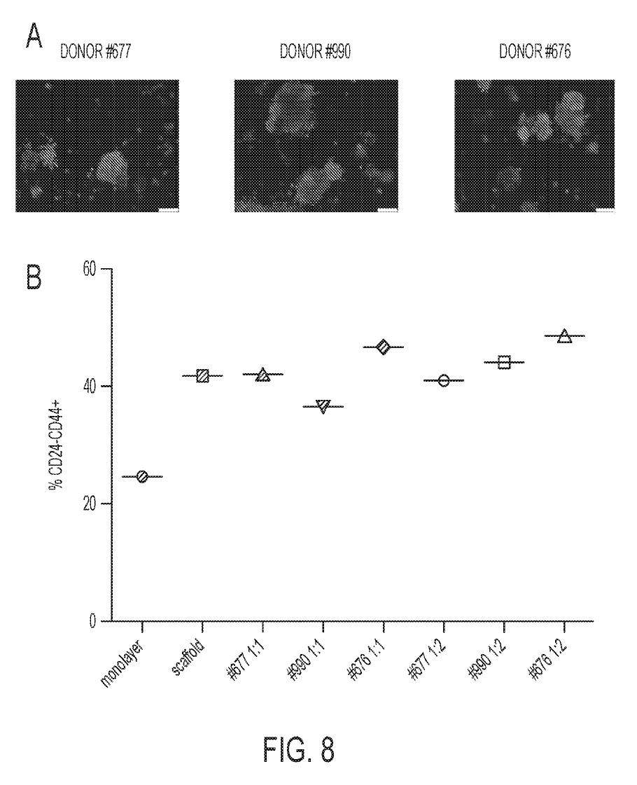

MDSCs isolated from three different individuals were o-cultured with MCF-7 cells on a scaffold (FIG. 8A). The co-culture formed irregular tumoroids that were slightly larger in size than with MCF-SCTs and the CD44⁺ CD24⁻ stem cell-like population showed a slight increase versus MCF-SCTs (FIG. 8B). However, we also observed variation in the fold induction between the three donor MDSCs used.

### Example 9. FiSS Induces Cancer Stem Cell Expansion

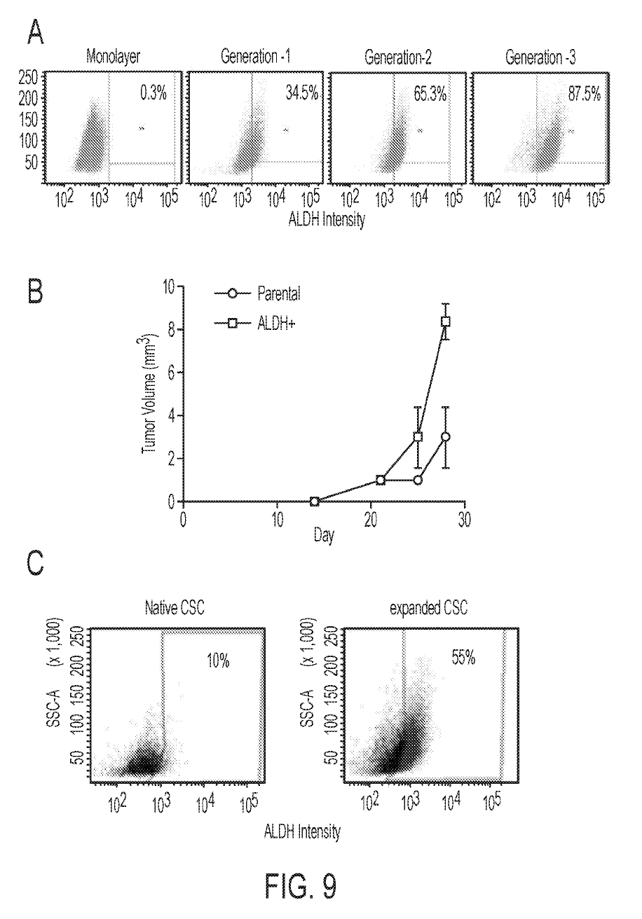

We found that culturing LLC1 on the FiSS platform induced at least a 30-fold increase in CSC activity, identified based on aldehyde dehydrogenase (ALDH) activity, as compared to monolayer cells (0.3%) (FIG. 9A). These tumoroids exhibited an ALDH-Hi population, representing a CSC-like population compared to cells grown on monolayer. The sorted ALDH-positive population could initiate better tumor growth in C57BL/6 mice than the unsorted LLC1 tumoroids (FIG. 9B), implying that the ALDH-positive population possesses CSC-like cells. In addition, successive passaging of these tumoroids on FiSS enriched ALDH positive cells to 87.5% in the third generation and a majority of stemness genes were conserved in expanded populations. To examine the potential to isolate and expand the rare CSC population from in vivo tumors, we resected LLC1 tumors implanted in C57BL/6 mice. When single cell suspensions of LLC1 tumors were cultured on FiSS for 6 days, a 10-fold expansion of ALDH-positive population was found (FIG. 9C).

### Example 10. Characterization of CSCs in Xenografts Derived from A549 Lung Cancer Grown on FiSS^{CSC}

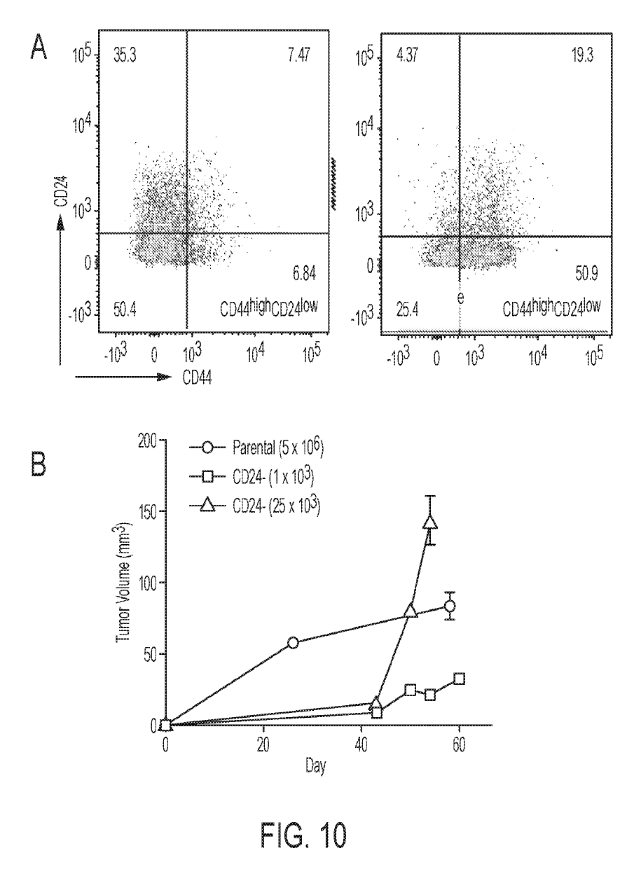

We next examined the potential to isolate and expand the rare CSC population from in vivo tumors. To examine the potential to isolate and expand the rare CSC population from in vivo tumors, we resected LLC1 tumors implanted in C57BL/6 mice. When single cell suspensions of LLC1 tumors were cultured on FiSS for 6 days, a 10-fold expansion of ALDH-positive population was found. Similarly, A549 xenografts implanted in NSG mice and single cell suspensions of these tumors were cultured on FiSS for 6 days. A 5- to 9-fold increase in CD44⁺ CD24⁻ population representing stem-like cells were found in tumoroids derived from A549 xenograft cells cultured on FiSS (50.9%) compared to in A549 xenografts (6.84%) (FIG. 10A). Moreover, injection of at least 1,000 CD44⁺ CD24⁻ population in NSG mice could initiate tumors in vivo, suggesting that CD44+ CD24− cells truly represent CSC-like cells in A549. Thus, the evidence that compared to primary injection of 3×10⁶ cells, injection of only 20,000 CD44+ CD24− cells induced the same size of tumor, indicates that the CSC expansion protocol we have developed enriches for tumor initiating cells (FIG. 10B).

We obtained ^{˜}10⁶ cells per A549 xenograft and after culturing on one 6-well format FiSS^{CSC} plate; we enriched by ^{˜}50% the cells expressing CD44⁺ CD24⁻. Thus, it is possible to collect ^{˜}10⁷ CD44⁺ CD24⁻ cells from v20 A549 xenografts. Similar strategies can be used isolate CD44⁺ CD24⁻ CSC-like cells from other xenografts.

### Example 11. Storage of Purified Cancer Stem Cells

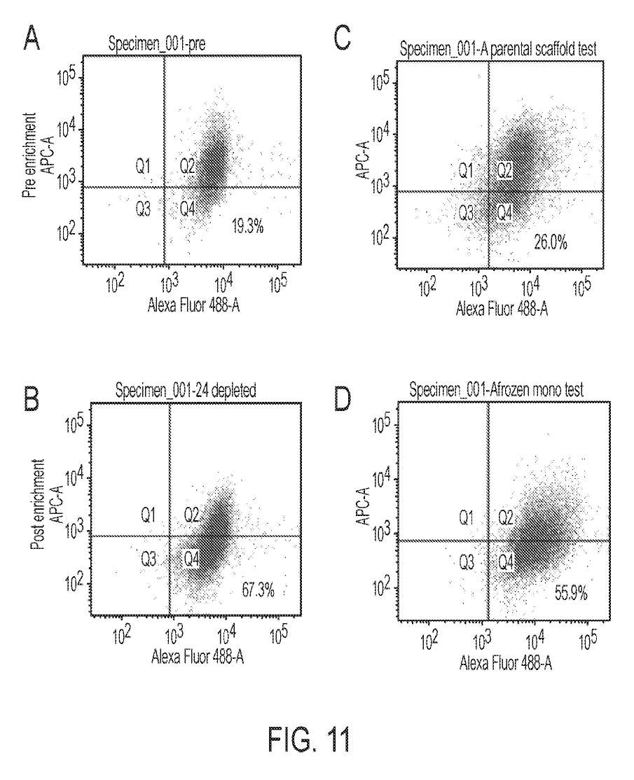

To examine whether the expanded CSCs could be stored appropriately, we used microbeads and processed CD44⁺ cells and then froze these cells in Cryostor® medium, as an example. Prior to enrichment, in the monolayer cultured cells, 19% of cells were CD44⁺ whereas of the cells grown on the scaffold, ^{˜}26% were CD44⁺. Furthermore, after depletion of tumoroid cells, 67% were CD44⁺ cells. After freezing and thawing the cells, ^{˜}55% of the cells were CD44⁺ (FIG. 11).

Embodiments of the present invention include at least the following. In one embodiment of the present invention, regular growth medium was used to grow first-generation MCF-7 tumoroids, and second-generation tumoroids were grown from the first-generation MCF-7 tumoroids. At the end of each generation, we processed the resulting tumoroids and analyzed the cells for stem cell markers (e.g., CD44^{high} and CD24^{low}) by flow cytometry. The results showed that the first-generation MCF-7 tumoroids gave a ^{˜}3-fold increase in CSCs.

In another embodiment, regular growth medium was used supplemented with cobalt chloride to mimic hypoxia in the first-generation MCF-7 tumoroids. Alternatively, cobalt chloride was infused into the scaffold matrix to ensure sustained hypoxic conditions for first-generation MCF-7 tumoroids growing on the scaffold. This increase was further potentiated in the second-generation tumoroids, where we observed a ^{˜}10-fold increase in CSCs. While supplementing with cobalt chloride had little effect on CSC amplification, growing first-generation tumoroids on cobalt chloride-infused scaffolds gave us larger first-generation tumoroids that showed a trend towards increased CSCs compared with tumoroids grown on regular scaffolds.

In a further embodiment, the CSC population was further increased by culturing the tumoroids in conditioned media (CM) collected from primary cancer-associated fibroblasts (CAFs) and myeloid-derived suppressor cells (MDSCs) from human peripheral blood.

In another embodiment, tumoroid culture conditions were expanded from a 96-well format to a six-well format tissue culture dish to increase the yield of CSCs (by 30-fold), while maintaining the ability for CSC expansion.

In yet another embodiment, long-term storage and tests of viability and functional properties of CSCs were examined, the results of which demonstrated the feasibility of stem cell expansion and storage.

Other embodiments of the present invention include at least the following:

An in vitro method for cancer stem cell (CSC) expansion comprising growing tumoroids on a scaffold and separating CSCs from the culture.

An in vitro method for cancer stem cell (CSC) expansion comprising growing tumoroids on a scaffold and separating CSCs from the culture, wherein said tumoroids are cultured in medium comprising conditioned medium (CM) collected from primary human cancer-associated fibroblasts (CAFs).

An in vitro method for cancer stem cell (CSC) expansion comprising growing tumoroids on a scaffold and separating CSCs from the culture, wherein said tumoroids are cultured in medium comprising conditioned medium (CM) collected from primary myeloid-derived suppressor cells (MDSCs) from human peripheral blood.

An in vitro method for cancer stem cell (CSC) expansion comprising growing tumoroids on a scaffold and separating CSCs from the culture, wherein said scaffold is a fiber-inspired smart scaffold (FiSS™).

An in vitro method for cancer stem cell (CSC) expansion comprising growing tumoroids on a scaffold and separating CSCs from the culture, wherein said scaffold further comprises and ECM-based hydrogel. In one embodiment, the ECM-based hydrogel is a solubilized basement membrane preparation extracted from the Engelbreth-Holm-Swarm (EHS) mouse sarcoma, a tumor rich in such ECM proteins as laminin (a major component), collagen IV, heparin sulfate proteoglycans, entactin/nidogen, and growth factors (e.g., MATRIGEL® by Corning Life Sciences and BD Biosciences or CULTREX® Basement Membrane Extract (BME) by Trevigen Inc.).

An in vitro method for cancer stem cell (CSC) expansion comprising growing tumoroids on a scaffold and separating CSCs from the culture, wherein said CSCs are harvested from first-generation tumoroids.

An in vitro method for cancer stem cell (CSC) expansion comprising growing tumoroids on a scaffold and separating CSCs from the culture, wherein said CSCs are harvested from second-generation tumoroids, grown from first-generation tumoroids.

An in vitro method for cancer stem cell (CSC) expansion comprising growing tumoroids on a scaffold and separating CSCs from the culture, wherein said CSCs are harvested from third-generation tumoroids, grown from second-generation tumoroids, grown from first-generation tumoroids.

A scaffold for cancer stem cell (CSC) expansion comprising a mixture of poly(lactic-co-glycolic acid) (PLGA) and a block copolymer of polylactic acid (PLA) and monomethoxypolyethylene glycol (mPEG).

A scaffold for cancer stem cell (CSC) expansion comprising a mixture of poly(lactic-co-glycolic acid) (PLGA) and a block copolymer of polylactic acid (PLA) and monomethoxypolyethylene glycol (mPEG), wherein said scaffold is prepared by electrospinning said mixture of poly(lactic-co-glycolic acid) (PLGA) and a block copolymer of polylactic acid (PLA) and monomethoxypolyethylene glycol (mPEG).

A scaffold for cancer stem cell (CSC) expansion comprising a mixture of poly(lactic-co-glycolic acid) (PLGA) and a block copolymer of polylactic acid (PLA) and monomethoxypolyethylene glycol (mPEG), wherein said scaffold further comprises cobalt chloride (CoCl₂).

A scaffold for cancer stem cell (CSC) expansion comprising a mixture of poly(lactic-co-glycolic acid) (PLGA) and a block copolymer of polylactic acid (PLA) and monomethoxypolyethylene glycol (mPEG), wherein said scaffold is a fiber-inspired smart scaffold (FiSS™).

A scaffold for cancer stem cell (CSC) expansion comprising a mixture of poly(lactic-co-glycolic acid) (PLGA) and a block copolymer of polylactic acid (PLA) and monomethoxypolyethylene glycol (mPEG), wherein said scaffold further comprises Matrigel®.

A scaffold for cancer stem cell (CSC) expansion comprising a mixture of poly(lactic-co-glycolic acid) (PLGA) and a block copolymer of polylactic acid (PLA) and monomethoxypolyethylene glycol (mPEG), wherein said scaffold induces hypoxic culture conditions.

A scaffold for cancer stem cell (CSC) expansion comprising a mixture of poly(lactic-co-glycolic acid) (PLGA) and a block copolymer of polylactic acid (PLA) and monomethoxypolyethylene glycol (mPEG), wherein said scaffold further comprises cobalt chloride (CoCl₂).

A scaffold for cancer stem cell (CSC) expansion comprising a mixture of poly(lactic-co-glycolic acid) (PLGA) and a block copolymer of polylactic acid (PLA) and monomethoxypolyethylene glycol (mPEG), wherein said scaffold further comprises one or more iron chelators.

A scaffold for cancer stem cell (CSC) expansion comprising a mixture of poly(lactic-co-glycolic acid) (PLGA) and a block copolymer of polylactic acid (PLA) and monomethoxypolyethylene glycol (mPEG), wherein said scaffold further comprises a siRNA that knocks down the von Hippel-Lindau (VHL) tumor suppressor gene.

A scaffold for cancer stem cell (CSC) expansion comprising a mixture of poly(lactic-co-glycolic acid) (PLGA) and a block copolymer of polylactic acid (PLA) and monomethoxypolyethylene glycol (mPEG), wherein said scaffold further comprises DNA encoding growth factors.

A scaffold for cancer stem cell (CSC) expansion comprising a mixture of poly(lactic-co-glycolic acid) (PLGA) and a block copolymer of polylactic acid (PLA) and monomethoxypolyethylene glycol (mPEG), wherein said scaffold further comprises TGF-β.

An in vivo method for cancer stem cell (CSC) expansion comprising growing a xenograft in a host animal by injecting cancer cells, separating cells from the recovered xenograft, growing them on a scaffold to form tumoroids, and separating CSCs from the culture.

An in vivo method for cancer stem cell (CSC) expansion comprising growing a xenograft in a host animal by injecting cancer cells, separating cells from the recovered xenograft, growing them on a scaffold to form tumoroids, and separating CSCs from the culture, wherein said host animal is a mouse.

An in vivo method for cancer stem cell (CSC) expansion comprising growing a xenograft in a host animal by injecting cancer cells, separating cells from the recovered xenograft, growing them on a scaffold to form tumoroids, and separating CSCs from the culture, wherein said host animal is a NOD-EGFP mouse.

An in vivo method for cancer stem cell (CSC) expansion comprising growing a xenograft in a host animal by injecting cancer cells, separating cells from the recovered xenograft, growing them on a scaffold to form tumoroids, and separating CSCs from the culture, wherein said cancer cells are human cancer cells.

An in vivo method for cancer stem cell (CSC) expansion comprising growing a xenograft in a host animal by injecting cancer cells, separating cells from the recovered xenograft, growing them on a scaffold to form tumoroids, and separating CSCs from the culture, wherein said cancer cells are injected with Matrigel®.

A method for in vitro cancer stem cell (CSC) expansion comprising growing tumoroids on a scaffold and separating CSCs from the culture.

A method for in vitro cancer stem cell (CSC) expansion comprising growing tumoroids on a scaffold and separating CSCs from the culture, wherein said scaffold is a fiber-inspired smart scaffold (FiSS™).

A method for in vitro cancer stem cell (CSC) expansion comprising growing tumoroids on a scaffold and separating CSCs from the culture, wherein said scaffold is prepared by electrospinning a mixture of poly(lactic-co-glycolic acid) (PLGA) and a block copolymer of polylactic acid (PLA) and monomethoxypolyethylene glycol (mPEG).

A method for in vitro cancer stem cell (CSC) expansion comprising growing tumoroids on a scaffold and separating CSCs from the culture, wherein said scaffold further comprises cobalt chloride (CoCl₂).

A method for in vitro cancer stem cell (CSC) expansion comprising growing tumoroids on a scaffold and separating CSCs from the culture, wherein said tumoroids are cultured in medium comprising conditioned medium (CM) collected from primary human cancer-associated fibroblasts (CAFs).

A method for in vitro cancer stem cell (CSC) expansion comprising growing tumoroids on a scaffold and separating CSCs from the culture, wherein said tumoroids are cultured in medium comprising Matrigel®.

A method for in vitro cancer stem cell (CSC) expansion comprising growing tumoroids on a scaffold and separating CSCs from the culture, wherein said CSCs are harvested from first-generation tumoroids.

A method for in vitro cancer stem cell (CSC) expansion comprising growing tumoroids on a scaffold and separating CSCs from the culture, wherein said CSCs are harvested from second-generation tumoroids, grown from first-generation tumoroids.

A method for in vitro cancer stem cell (CSC) expansion comprising growing tumoroids on a scaffold and separating CSCs from the culture, wherein said CSCs are harvested from third-generation tumoroids, grown from second-generation tumoroids, grown from first-generation tumoroids.

A method for in vitro cancer stem cell (CSC) expansion comprising growing tumoroids on a scaffold and separating CSCs from the culture, wherein said scaffold induces hypoxic culture conditions.

A method for in vitro cancer stem cell (CSC) expansion comprising growing tumoroids on a scaffold and separating CSCs from the culture, wherein said scaffold further comprises one or more iron chelators.

A method for in vitro cancer stem cell (CSC) expansion comprising growing tumoroids on a scaffold and separating CSCs from the culture, wherein said scaffold further comprises a siRNA that knocks down the von Hippel-Lindau (VHL) tumor suppressor gene.

A method for in vitro cancer stem cell (CSC) expansion comprising growing tumoroids on a scaffold and separating CSCs from the culture, wherein said scaffold further comprises DNA encoding growth factors.

A method for in vitro cancer stem cell (CSC) expansion comprising growing tumoroids on a scaffold and separating CSCs from the culture, wherein said scaffold further comprises TGF-β.

A method for in vivo cancer stem cell (CSC) expansion comprising growing a xenograft in a host animal by injecting cancer cells, separating cells from the recovered xenograft, growing them on a scaffold to form tumoroids, and separating CSCs from the culture.

A method for in vivo cancer stem cell (CSC) expansion comprising growing a xenograft in a host animal by injecting cancer cells, separating cells from the recovered xenograft, growing them on a scaffold to form tumoroids, and separating CSCs from the culture, wherein said cancer cells are obtained from a mammal.

A method for in vivo cancer stem cell (CSC) expansion comprising growing a xenograft in a host animal by injecting cancer cells, separating cells from the recovered xenograft, growing them on a scaffold to form tumoroids, and separating CSCs from the culture, wherein said cancer cells are obtained from a mammal, wherein said mammal is an experimental animal model of a cancer.

A method for in vivo cancer stem cell (CSC) expansion comprising growing a xenograft in a host animal by injecting cancer cells, separating cells from the recovered xenograft, growing them on a scaffold to form tumoroids, and separating CSCs from the culture, wherein said cancer cells are obtained from a mammal, wherein said mammal is an experimental animal model of a human cancer.

A method for in vivo cancer stem cell (CSC) expansion comprising growing a xenograft in a host animal by injecting cancer cells, separating cells from the recovered xenograft, growing them on a scaffold to form tumoroids, and separating CSCs from the culture, wherein said cancer cells are from a human biopsy.

A method for in vivo cancer stem cell (CSC) expansion comprising growing a xenograft in a host animal by injecting cancer cells, separating cells from the recovered xenograft, growing them on a scaffold to form tumoroids, and separating CSCs from the culture, wherein said cancer cells are human tumor cells.

A method for in vivo cancer stem cell (CSC) expansion comprising growing a xenograft in a host animal by injecting cancer cells, separating cells from the recovered xenograft, growing them on a scaffold to form tumoroids, and separating CSCs from the culture, wherein said cancer cells are injected with Matrigel®.

Unless indicated otherwise, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained with the present invention. At the very least, and not as an attempt to limit the application of the doctrine of equivalents to the scope of the claims, each numerical parameter should at least be construed in light of the number of reported significant digits and by applying ordinary rounding techniques.

While the invention has been particularly shown and described with reference to preferred embodiments, it will be understood by those skilled in the art that various changes in form and detail may be made therein without departing from the spirit and scope of the invention.

All documents, publication, manuals, article, patents, summaries, references, and other materials cited here are incorporated by reference in their entirety. Other embodiments of the invention will be apparent to those skilled in the art from consideration of the specification and practice of the invention disclosed here. It is intended that the specification and examples be considered as exemplary, with the true scope and spirit of the invention indicated by the claims.

The claimed invention is:

1. A method for expanding cancer stem cells (CSCs) comprising:
a) growing tumoroids on a three-dimensional scaffold in an in vitro cell culture; and,
b) isolating CSCs from said tumoroids.

2. A method for expanding cancer stem cells (CSCs) comprising:
a) growing cancer cells in an in vitro cell culture comprising a three-dimensional scaffold;
b) growing tumoroids from said cancer cells on said scaffold;
c) harvesting cancer cells from said tumoroids (tumoroid cancer cells);
d) transferring said tumoroid cancer cells to a new in vitro cell culture comprising a three-dimensional scaffold;
e) growing a subsequent generation of tumoroids from said tumoroid cancer cells on said scaffold of said new in vitro cell culture.

3. The method of claim 2, wherein steps c) through e) are repeated at least once.

4. The method of claim 2, wherein said steps c) through e) are repeated at least twice, at least three times, at least four times, at least five times, at least six times, or at least seven times.

5. The method of any one of claims 2-4, comprising: isolating CSCs from said tumoroids.

6. The method of any one of claims 1-5, wherein said method comprises: dissociating said tumoroids single cells or tumoroid cell fragments of less than 1,000, 500, 100, 50, or 10 cells.

7. The method of any one of claims 1-6, wherein said method comprises: forming a single-cell suspension of tumoroid cancer cells from said tumoroids.

8. The method of any one of claims 1, or 5-7, wherein said isolating CSCs from said tumoroids comprises: forming a single-cell suspension of tumoroid cancer cells from said tumoroids; and, isolating CSCs from said single-cell suspension of said tumoroid cancer cells.

9. The method of any one of the preceding claims, wherein said method comprises growing tumoroids from human cancer cells.

10. The method of claim 9, wherein said human cancer cells are from a human biopsy.

11. The method of any one of the preceding claims, wherein said scaffold comprises an electrospun mixture of poly(lactic-co-glycolic acid) (PLGA) and a block copolymer of polylactic acid (PLA) and monomethoxypolyethylene glycol (mPEG).

12. The method any one of the preceding claims, wherein said tumoroids are grown in hypoxic conditions or conditions that mimic hypoxic conditions, throughout the cell culture or local to the scaffold.

13. The method of claim 12, wherein said cell culture or scaffold further comprises cobalt chloride (CoCl₂).

14. The method of claim 13, wherein said CoCl₂ is added to a mixture of poly(lactic-co-glycolic acid) (PLGA) and a block copolymer of polylactic acid (PLA) and monomethoxypolyethylene glycol (mPEG) prior to electrospinning.

15. The method of any one of the preceding claims, wherein said tumoroids are cultured in medium comprising conditioned medium (CM) collected from: primary human cancer-associated fibroblasts (CAFs) and/or myeloid-derived suppressor cells (MDSCs) from human peripheral blood.

16. The method of any one of the preceding claims, wherein said tumoroids are cultured in medium comprising an ECM-based hydrogel.

17. The method of claim 16, wherein said ECM-based hydrogel is a solubilized basement membrane preparation extracted from the Engelbreth-Holm-Swarm (EHS) mouse sarcoma.

18. The method of any one of the preceding claims, wherein a first-generation of said tumoroids have at least a 2-fold, 2.5-fold, or 3-fold increase in CSCs compared to cancer cells used to grow the first-generation tumoroids.

19. The method of any one of the preceding claims, wherein said method comprises growing a second-generation of tumoroids, and wherein said second-generation of tumoroids have at least a 5-fold, 6-fold, 7-fold, 8-fold, 9-fold, or 10-fold increase in CSCs compared to the first-generation tumoroid cancer cells used to grow the second-generation tumoroids or to the cancer cells used to grow the first-generation tumoroids.

20. The method of claim 19, wherein said second-generation tumoroids have at least a 10-fold increase in CSCs compared to the first-generation tumoroid cancer cells used to grow the second-generation tumoroids.

21. The method of claim 20, wherein said second-generation tumoroids have at least a 10-fold, 15-fold, 20-fold, 25-fold, 30-fold, 40-fold, 50-fold, 60-fold, 70-fold, 75-fold, or 80-fold increase in CSCs compared to the cancer cells used to grow the first-generation tumoroids.

22. The method of claim 21, wherein said second-generation tumoroids have at least an 80-fold increase in CSCs compared to the cancer cells used to grow the first-generation tumoroids.

23. The method of any one of the preceding claims, wherein said method comprises growing a third-generation of tumoroids, and wherein said third-generation of tumoroids have at least a 10-fold, 15-fold, 20-fold, 25-fold, 30-fold, 40-fold, 50-fold, 60-fold, 70-fold, 75-fold, or 80-fold increase in CSCs compared to: the cancer cells used to grow the first-generation tumoroids, the first-generation tumoroids, or the second-generation tumoroids.

24. The method of claim 19, wherein said method comprises growing a third-generation of tumoroids, and wherein said third-generation of tumoroids have at least a 10-fold, 15-fold, 20-fold, 25-fold, 30-fold, 40-fold, 50-fold, 60-fold, 70-fold, 75-fold, or 80-fold increase in CSCs compared to the cancer cells used to grow the first-generation tumoroids.

25. The method of any one of the preceding claims, wherein said CSCs are harvested from a first-, second-, third-, or fourth- fifth-, sixth-, seventh-, eighth-, ninth- or tenth-generation tumoroids.

26. The method of any one of the preceding claims, wherein said CSCs isolated from said tumoroids are used to grow the next generation of tumoroids.

27. The method of any one of the preceding claims, wherein said CSCs isolated from first-generation tumoroids are used to grow second-generation tumoroids.

28. The method of any one of the preceding claims, wherein said CSCs isolated from second-generation tumoroids are used to grow third-generation tumoroids.

29. The method of any one of the preceding claims, wherein said CSCs isolated from first-generation tumoroids are used to grow second-generation tumoroids and CSCs isolated from said second-generation tumoroids are used to grow third-generation tumoroids.

30. The method of any one of the preceding claims, wherein said culture or scaffold further comprises one or more iron chelators.

31. The method of claim 30, wherein said one or more iron chelators is added to a mix of said poly(lactic-co-glycolic acid) (PLGA) and a block copolymer of polylactic acid (PLA) and monomethoxypolyethylene glycol (mPEG) prior to electrospinning.

32. The method of any one of the preceding claims, wherein said culture or scaffold further comprises a siRNA that knocks down von Hippel-Lindau (VHL) tumor suppressor gene.

33. The method of any one of the preceding claims, wherein said cancer cells comprise a heterologous DNA encoding a growth factor.

34. The method of any one of the preceding claims, wherein said culture or scaffold further comprises TGF-β.

35. A method for cancer stem cell (CSC) expansion comprising:
a) injecting cancer cells into a non-human host animal to form a tumor;
b) removing said tumor from said host animal;
c) dissociating said tumor into a suspension of tumor cells and/or tumor fragments; and,
d) growing said suspension on a three-dimensional scaffold in an in vitro cell culture to form tumoroids.

36. The method of any one of claims 1-34, wherein said method comprises:
a) injecting cancer cells into a non-human host animal to form a tumor;
b) removing said tumor from said host animal;
c) dissociating said tumor into a suspension of tumor cells or tumor fragments; and,
d) growing said suspension on a three-dimensional scaffold in an in vitro cell culture to form tumoroids.

37. The method of claim 35 or 36, wherein said tumor is a tumor xenograft.

38. The method of claim 35 or 36, wherein said cancer cells are obtained from a mammal.

39. The method of any one of claims 35-38, wherein said cancer cells are from a human biopsy.

40. The method of any one of claims 35-39, wherein said cancer cells are human tumor cells.

41. The method of any one of claims 35-40, wherein said cancer cells are co-injected with ECM-based hydrogel.

42. The method of claim 35 or 36, wherein said tumoroids grown in step d) are first-generation tumoroids, second-generation tumoroids, third-generation, or fourth-generation tumoroids.

43. The method of claim 42, wherein said tumoroids are first-generation tumoroids.

44. The method of any one of the preceding claims, wherein said tumoroids are cultured in regular media.

45. A method of screening an anti-cancer drug compound comprising: a) culturing said tumoroids of any one of claims 1-44; b) contacting said tumoroids with an anti-cancer drug compound; and c) measuring an effect of said drug compound on said tumoroids.

46. A method of screening an anti-cancer drug compound comprising: a) culturing said tumoroid cancer cells of any one of claims 1-44; b) contacting said tumoroid cancer cells with an anti-cancer drug compound; and c) measuring an effect of said drug compound on said tumoroid cancer cells.

47. A method of screening an anti-cancer drug compound comprising: a) culturing said isolated CSCs of any one of claims 1-44; b) contacting said isolated CSCs with an anti-cancer drug compound; and c) measuring an effect of said drug compound on said isolated CSCs.

48. The method of claim 45, 46 or 47, wherein said method comprises measuring an IC₅₀, GI₅₀, ED₅₀ or LD₅₀ of said drug compound.
